(19) United States
(12) Patent Application Publication
(10) Pub. No.: US 2005/0260155 A1
(43) Pub. Date: Nov. 24, 2005

(54) Compositions and methods for treatment of ulcerative colitis

(76) Inventors: Gopala Kovvali, Edison, NJ (US); Kiron M. Das, Basking Ridge, NJ (US)

Correspondence Address: Jane Massey Licata;Licata & Tyrrell P.C., 66 E. Main Street, Marlton, NJ, 08053, US

(21) Appl. No.: 11/132,104

(22) Filed: May 18, 2005

Related U.S. Application Data
(60) Provisional application No. 60/572,329, filed on May 18, 2004.

(51) Int. Cl. A61K 38/21; G01N 33/53; A61K 38/17

(52) U.S. Cl. 424/85.6; 514/12; 435/7.1

(57) ABSTRACT

The present invention relates to the identification of TCP-1 gamma as a protein whose expression is decreased in patients with ulcerative colitis and Crohn's disease. This protein interacts with hTM5, which is involved in the pathogenesis of ulcerative colitis. Compositions and methods for diagnosis and treatment, including methods for following up the efficacy of treatment of ulcerative colitis are provided.

### INTRODUCTION

This application claims the benefit of priority from U.S. provisional application Ser. No. 60/572,329, filed May 18, 2004, the contents of which are incorporated by reference herein in its entirety.

### BACKGROUND OF THE INVENTION

Tropomyosins are microfilament-associated proteins present in all eukaryotic cells with organ specific isoforms and distinct functions (Lees-Miller et al. 1991. BioEssays 13:429-437; Pittenger et al. 1992. J. Cell Biol. 118:841-858; Lin et al. 1997. Int. Rev. Cytol. 170:1-38). A human fibroblast cell line expresses at least eight tropomyosin isoforms termed hTM1, hTM2, hTM3, hTMsmα, hTM5a, hTM5b, hTM4, and hTM5, which are encoded by four different genes (Lin et al. 1997. Int. Rev. Cytol. 170:1-38; Novy et al. 1993. Cell Motil. Cytoskel. 25:267-281). The tropomyosin molecule is almost a fully α-helical protein with multiple heptad repeats and capable of forming a coiled core dimer (Smillie. 1979. Trends Biochem. Sci. 4:151-155). These features appear to be associated with many known autoantibody epitopes and may contribute to autoantigenic potential (Dohlman et al. 1993. Biochem. Biophys. Res. Commun. 195:686-696). Several observations suggest that tropomyosin is an autoantigen for ulcerative colitis (Das et al. 1993. J. Immunol. 150:2487-2493; Biancone et al. 1995. Gastroenterology 109:3-12; Geng et al. 1998. Gastroenterology 114:912-922; Sakamaki et al. 2000. Gut 47:236-241; Mizoguichi et al. 1996. J. Exp. Med. 183:847-856).

Ulcerative colitis is a chronic inflammatory bowel disease characterized by inflammation and ulceration of the colon mucosa. Autoimmunity has been suggested as important to the pathogenesis of ulcerative colitis, where the effects are an autoimmune response to cellular antigens. Such cellular antigens may include the tropomyosins. Autoantibodies against tropomyosins are found in sera of individuals with ulcerative colitis and in IgG produced by the cultured lamina propria mononuclear cells that infiltrate inflamed ulcerative colitis tissue. Furthermore, autoantibody to tropomyosin is found in a mouse model for human ulcerative colitis that has been created by targeting deletion of the T-cell receptor α (TCRα) gene (Mizoguchi et al. 1996. J. Exp. Med. 183:847-856). More recent studies using tropomyosin isoform-specific monoclonal antibodies have demonstrated that colonic epithelial cells synthesize two of the major tropomyosin isoforms, hTM4 and hTM5, whereas colonic smooth muscle cells contain at least he hTM1, hTM2, and hTM3 isoforms (Geng et al. 1998. Gastroenterology 114:912-922). Using recombinant tropomyosin isoforms it was also shown that ulcerative colitis patients produce significant autoantibodies, preferentially against hTM5 (Geng et al. 1998. Gastroenterology 114:912-922; Onuma et al. 2000. Clin. Exp. Immunol. 121:466-471). In fact, 42% of colonic mucosal lymphocytes were committed to production of anti-hTM5 antibody (Onuma et al. 2000. Clin. Exp. Immunol. 121:466-471). In studies in peripheral blood lymphocytes, a T cell response against hTM5 in ulcerative colitis, but not in Crohn's disease, has been demonstrated (Taniguichi et al. 2001. Clin. Immunol. 101:289-295).

These data indicate that immune responses, both by B and T cells, are involved in the immunopathogenesis of ulcerative colitis. This is further supported by research showing that hTM5 is expressed on the surface of human colonic epithelial cells but not on small intestine enterocytes, although both colonic and small intestine enterocytes have intracellular hTM5 (Kesari et al. 1999. Clin. Exp. Immunol. 118:219-227; Gneg et al. 1998. Gastroenterology 114:912-922). The mechanism by which hTM5 acts in the pathogenesis of ulcerative colitis remains to be elucidated.

### SUMMARY OF THE INVENTION

An object of the present invention is a composition for treating ulcerative colitis which comprises interferon gamma, TCP-1 gamma and a pharmaceutically acceptable vehicle.

Another object of the present invention is a method for decreasing the symptoms of ulcerative colitis in a patient which comprises contacting cells or tissues of a patient with the composition of the present invention, and monitoring the level of TCP-1 gamma gene expression in cells or tissues, wherein an increase in the expression of TCP-1 gamma in said cells or tissues will result in a decrease in the symptoms of ulcerative colitis in said patient.

Another object of the present invention is a method for diagnosing ulcerative colitis in a patient which comprises determining a level of TCP-1 gamma in lymphocytes from blood of a patient suspected of having ulcerative colitis; determining a level of TCP-1 gamma in lymphocytes from the blood of several healthy individuals; and comparing the magnitude of the levels of TCP-1 gamma in said patient and said healthy control individuals, wherein a decrease in the magnitude of the level of TCP-1 gamma in the patient as compared to the levels of the healthy control individuals is diagnostic of ulcerative colitis.

Yet another object of the present invention is a method for identifying compounds useful for treating ulcerative colitis which comprises harvesting lymphocytes from the blood of patients with ulcerative colitis; determining a first level of TCP-1 gamma in the lymphocytes; contacting the lymphocytes with a compound; and determining a second level of TCP-1 gamma activity in the lymphocytes, wherein the compound is identified as being useful of treating ulcerative colitis if the magnitude of the second level of TCP-1 gamma is greater than the magnitude of the first level of TCP-1 gamma.

An object of the present invention is a method for evaluating efficacy of treatment of ulcerative colitis which comprises determining a level of TCP-1 gamma in lymphocytes from blood of a patient before administration of an agent to treat ulcerative colitis; administering to the patient an agent capable of treating ulcerative colitis; determining a level of TCP-1 gamma in lymphocytes from the blood of the patient after administration of the agent; and comparing the magnitude of the levels of TCP-1 gamma in said patient before and after administration of the agent, wherein an increase in the magnitude of the level of TCP-1 gamma in the patient after treatment with the agent as compared to the levels of TCP-1 gamma in lymphocytes before treatment with the agent is indicative of an effective treatment for ulcerative colitis.

### DETAILED DESCRIPTION OF THE INVENTION

The etiology of autoimmune disease has been linked to both internal and external factors. An internal factor would be some substance in the host's body that by its actions initiates events that lead to disease. An external factor would be some agent foreign to the host's body that triggers disease. Research has suggested that bacterial and viral infections may initiate or cause autoimmune disease or that they may be the factor that tilts the balance from tolerance to defense or attack of the host, a natural mechanism of self-preservation.

In the case of microbes, the central issue for autoimmune disease would be whether the microbes initiate, propagate or accentuate the autoimmune response. There is some evidence to suggest that microbial cells or some substance produced by the microbe acts as a molecular mimic and thus interferes with a host's natural defense mechanisms, initiating disease. Molecular mimicry is a concept that states that antigenic determinants of infectious microorganisms resemble structures in the tissue of the host but differ enough to be recognized as foreign by the host immune system (Rose. 2001. J. Clin. Invest. 107:943-944). Support for the concept of molecular mimicry as a mechanism active in autoimmune disease is found in the work showing that antibodies against the phosphoprotein of measles virus and Herpes simplex type I cross-reacted with an intermediated filament protein, vimentin, in human cells (Fujinami et al. 1983. Proc. Natl. Acad. Sci. USA 80:2346-2350).

Ulcerative colitis has been linked to an autoimmune pathogenesis. Research indicates that there is not likely to be one single etiological factor responsible for the onset of disease. However, the fact that ulcerative colitis patients are prone to infection suggests a role for bacteria or viruses in the disease process. Ulcerative colitis, unlike bacterial colitis, is a chronic disease, and it has been suggested that the host immune system initiates a response due to signals produced by bacteria.

It has already been established that a cytoplasmic protein, hTM5, is expressed in colon cells and is a potential auto-antigen in a subset of ulcerative colitis patients (Onuma et al. 2000. Clin. Exp. Immunol. 121:466-471). It is not understood, however, what the role of this protein is in the disease process and whether it is some characteristic of the protein itself that is critical for disease process initiation or promulgation. With the fact that bacteria may also be involved in some part of the disease process, under at least some circumstances, experiments were performed to look for homology between hTM5 and bacterial genomes. The basis for these experiments was to determine if there is some type of similarity in hTM5 and the bacteria that could form the basis of a molecular mimicry that is involved in the autoimmune disease process. The importance of sequence homology to molecular mimicry has been defined (Kovvali and Das. 2005. FEBS Letters 579:2261-2266).

Through searching of the NCBI database, two bacterial proteins were identified that had regions of significant sequence homology with hTM5. One of the identified bacterial proteins was a sensor protein from Bacillus cereus (Accession No. NP_{—}834548). The other is a hypothetical protein from cyanobacteria Nosoc punctiforme (Accession No. ZP 00112434). While the sensor protein has homology with the C-terminal portion of hTM5, the other protein resembles the N-terminal portion of hTM5. Interestingly, only the N-terminal and C-terminal peptides of hTM5 are found to have significant homology with these bacterial proteins. Further, the sensor protein from Bacillus cereus is identical to the VanS sensor protein antracis. The presence of the sequence homology of these bacterial proteins with hTM5 indicates a role for these bacteria in the disease process of ulcerative colitis, in the same way that hTM5 has been implicated. The homology also points to a role for hTM5, and potentially other tropomyosins, as molecular mimics in the process of autoimmune disease, in particular ulcerative colitis. The data also suggest that the C-terminal portion of hTM5, located between amino acids 188-213, is a potential region involved in the molecular mimicry process of disease initiation.

Surface expression of hTM5 is believed to be involved in the pathogenesis of ulcerative colitis. It has now been found that the biochemical mechanism of the transport of hTM5 to the cell surface may be mediated via interactions with T complex polypeptide 1 (TCP-1), the first member of the TF55/TCP-1 family of chaperonins found in the family of chaperonins.

TCP-1 was found to interact with hTM5 in a two-hybrid screen. The two-hybrid library used in this analysis was generated by cloning cDNA from a colon cancer cell line, LS 180, into the activation domain vector pB42AD harboring a tryptophan (TRP) marker (MATCHMAKER™ LexA Two-hybrid system, CLONTECH™ Laboratories, CA). Full-length hTM5 cDNA was cloned into the binding domain of vector pGilda which has a histidine (HIS) marker. Yeast strain EGY 48, which has an integrated leucine 2 (LEU2) gene under the control of UAS, was used as the host strain. EGY48 cells were co-transformed with the hTM5 cloned binding domain plasmid and a lacZ reporter plasmid p-LacZ. Two-hybrid cDNA library was transformed with this strain and plated onto plates containing dextrose but lacking TRP, HIS and uracil (URA). Approximately one million transformants were collected into a tube by scraping the plates. Cells were plated on a galactose-containing but LEU-lacking medium to induce the expression of proteins from the binding domain and activation domain vectors. The colonies were thus screened for expression of LEU2 by monitoring expression of this gene on LEU-deprived plates. The colonies were further screened to identify cells containing library plasmids that produce proteins that interact with hTM5. Colonies growing on the LEU-deprived plates were transferred onto nitrocellulose membranes, permeabilized with liquid nitrogen, and incubated at 30° C. on plates containing x-gal. Colonies that turned blue were picked and the library plasmid rescued and sequenced. The NCBI database was searched against this DNA sequence and it was found that the gene sequence in the activation domain plasmid was that of TCP-1 gamma. Therefore, TCP-1 was identified as the protein that interacts with hTM5.

The interaction between hTM5 and TCP-1 gamma was verified using another type of assay. TCP-1 gamma gene containing activation domain plasmid and TH5 containing binding domain plasmid were re-transformed into EGY48 yeast cells. Several colonies were picked and patched on galactose-containing media or dextrose-containing media lacking LEU, TRP and histidine (HIS). The plates were incubated for two days at 30° C. The cells patched onto galactose-containing plates grew whereas those patched onto dextrose-containing plates did not grow. A frame-shift mutant generated during cloning of hTM5 cDNA into binding domain plasmid was used as a control to assess the genuine nature of the interaction between hTM5 and TCP-1 gamma. The mutant hTM5 did not interact with TCP-1 gamma, confirming that the interaction between TCP-1 gamma and hTM5 was a specific interaction. Two other non-epithelial isoforms of tropomyosin, TM3 and TM4, also failed to interact with TCP-1 gamma.

The next step was to determine if there was a specific region of hTM5 that binds to TCP-1 gamma. Experiments were performed that capitalized on the structural differences between hTM5 and the novel isoform TC22, which was previously cloned and sequenced (Lin et al. 2002. Gastroenterology 123:152-162). The amino acid sequence of TC22 isoform is identical to that of hTM5 except in the C-terminal domain, amino acids 222-247 coded by exon 9. TC22 expression is strongly associated with colonic neoplasia and carcinoma and is not expressed in normal epithelium (Lin et al. 2002. Gastroenterology 123:152-162). Using methods as described above, TCP-1 gamma was shown to interact with TC22 indicating that the domain of relevance in hTM5 for interaction with TCP-1 is within the first 221 amino acids.

With the earlier observation that hTM5 is secreted from colon cancer cells (LS 180) into the medium, experiments were performed to determine if TCP-1 gamma is also released from LS-180 cells. LS 180 cells were grown in serum free media using the methods previously described (Kesari et al. 1999. Clin. Exp. Immunol. 118:219-227). The presence of hTM5 and TCP-1 gamma was determined through western blot analysis using anti-hTM5 monoclonal antibody and anti-TCP-1 gamma polyclonal antibody. Both hTM5 and TCP-1 gamma were present in the supernatant of LS 180 cell culture. Since TCP-1 gamma is a member of a family of proteins known to as chaperone proteins, proteins that are essential for protein folding and often involved in transport of proteins across membranes, these data indicate a role for TCP-1 gamma specifically as a chaperone. Therefore, TCP-1 gamma was identified as a potential chaperone protein that transports hTM5 from cytosol to the cell surface and is then subsequently released from the cells with hTM5.

TCP-1 gamma is a 544 amino acid protein with a predicted molecular mass of 60 kDa. The protein in human has a high degree of structural homology with gamma subunits of mouse (98%) and yeast (Saccharomyces cerevisiae; 75%). With its known properties as a chaperone protein, the interaction with hTM5 indicates that TCP-1 gamma also acts as a chaperone for hTM5, thereby presenting hTM5 to the immune system as an antigen.

In order to further explore the role of TCP-1 gamma in ulcerative colitis, the expression of this chaperone protein in patients with ulcerative colitis was determined. The expression of TCP-1 gamma gene in total human lymphocytes was examined by semi-quantitative RT-PCR. RNA was isolated from peripheral blood mononuclear cells (PBMC) using RNEASY® micro kit according to the manufacturers instructions (QIAGEN®). RNA was eluted in a final volume of 18 μl. A portion of RNA (11 μl) was converted to first strand cDNA by reverse transcriptase reaction in 20 μl volume using random hexamers and MLV reverse transcriptase (INVITROGEN™ Inc., CA). Then, 9 μl of cDNA was used for PCR for each cytokine. PCR reactions were carried out in 50 μl for 35 cycles using Taq polymerase (INVITROGEN™, Inc., CA), while 4 μl of first-strand cDNA was used to amplify TCP-1 gamma and interferon-gamma (IFN-gamma) whereas 1 μl was used to amplify 18S RNA as a control. PCR products (18 μl) were analyzed on a 1.5% agarose gel. The amount of PCR products for each sample were compared with that for 18S. The ratio of intensity for TCP-1 gamma to that of 18S was quantified using KODAK® EDA290 gel documentation system. The ratio was a measure of the relative expression of the TCP-1 gamma gene.

Peripheral blood mononuclear cells were taken from 10 healthy subjects, 13 patients diagnosed with ulcerative colitis, and 9 patients diagnosed with Crohn's disease and analyzed using semi-quantitative polymerase chain reaction. The results of this analysis demonstrated that TCP-1 gamma was expressed in the PBMC of healthy subjects and its levels were higher than those in PBMCs of patients with ulcerative colitis. Based on statistical analysis (mean±SD) of healthy subjects, 8.0 was the cut off value to demarcate the relative expression levels of TCP-1 gamma in inflammatory bowel disease and healthy subjects. It was observed that 80% of healthy subjects had a ratio higher than 8.0, whereas 92% of ulcerative colitis patients have a ratio of less than 8.0 (Table 1, p<0.0001). Further, 66% of Crohn's disease patients also have the ratio less than 8.0.

TABLE 1

| | Relative Intensity of | Relative Intensity of |
|---|---|---|
| Subject | TCP-1 gamma Expression | IFN-gamma Expression |
| UC−1 | 3.8 | (−) |
| UC−2 | 2.8 | (−) |
| UC−3 | 7.8 | (−) |
| UC−4 | 14.9 | (−) |
| UC−5 | 3.7 | (−) |
| UC−6 | 1.6 | (−) |
| UC−7 | (−) | (−) |
| UC−8 | 8.3 | 13.8 |
| UC−9 | 1.57 | N/D |
| UC−10 | 3.4 | N/D |
| UC−11 | 1.6 | N/D |
| UC−12 | 3.02 | N/D |
| UC−13 | 3.33 | N/D |
| HS−1 | 9.6 | 30.75 |
| HS−2 | 28.7 | 44.6 |
| HS−3 | 19.9 | 22.44 |
| HS−4 | 21.9 | 15.1 |
| HS−5 | 18.01 | 14.05 |
| HS−6 | 13.8 | (−) |
| HS−7 | 10.35 | N/D |
| HS−8 | 8.53 | N/D |
| HS−9 | 6.22 | N/D |
| HS−10 | 6.81 | N/D |
| CR−l | 4.95 | N/D |
| CR−2 | 1.21 | N/D |
| CR−3 | 30.8 | 64.5 |
| CR−4 | 14.05 | 8.82 |
| CR−5 | 14.38 | 24.73 |
| CR−6 | | 6.06 |
| CR−7 | | (−) |
| CR−8 | 0.65 | 1.65 |
| CR−9 | 1.94 | 7.05 |

N/D, not determined.

The expression of IFN-gamma was also analyzed in some of the same samples (Table 1). Five out of six (83%) of the healthy subjects express IFN-gamma. whereas one out of eight ulcerative colitis (12.5%) patients express IFN-gamma endogenously.

It is contemplated that the expression of TCP-1 gamma in lymphocytes of healthy subjects confers protective effects and down-regulation of TCP-1 in ulcerative colitis may be associated with altered immune balance facilitating immune/autoimmune responses in inflammatory bowel disease. The data provided herein indicates a role of gamma interferon in regulating the expression of TCP-1 gamma in the lymphocytes of patients with inflammatory bowel disease. Accordingly, the present invention relates to the use of TCP-1 for treating ulcerative colitis and the use of TCP-1 as a marker for the diagnosis and prognosis of inflammatory bowel disease.

The present invention is a composition to treat ulcerative colitis which comprises a compound capable of increasing expression, activity or levels of TCP-1 gamma in cells or tissues. By increasing expression or activity or levels of TCP-1 gamma, such a composition would be capable of altering the activity of hTM5 in ulcerative colitis. In one embodiment the composition would comprise TCP-1 gamma administered in a pharmaceutically acceptable vehicle. Also contemplated by the present invention is any compound capable of either increasing expression of TCP-1 gamma gene or increasing the level or activity of TCP-1 gamma in cells or tissues. In a preferred embodiment, the compound would comprise interferon gamma and TCP-1 gamma.

The present invention is also a method for treating ulcerative colitis which comprises contacting cells or tissues with a compound capable of increasing expression, activity or levels of TCP-1 gamma in cells or tissues, wherein increasing the expression, levels or activity of TCP-1 gamma will result in a reduction in the activity of hTM5 and thus the symptoms of ulcerative colitis will be reduced. In one embodiment, the method of the present invention would involve administration of interferon gamma in conjunction with TCP-1 gamma.

The present invention is contemplated as a method for evaluating efficiency of treatment of ulcerative colitis. In this method, the expression of TCP-1 gamma can be measured in the lymphocytes of patients both before and after treatment is initiated and treatment efficacy would be demonstrated if the level of TCP-1 gamma expression is increased.

The present invention is a method for diagnosing ulcerative colitis in patients which comprises determining the level of TCP-1 gamma in lymphocytes from blood of patients suspected of having ulcerative colitis and comparing that level with the levels of TCP-1 gamma in lymphocytes from the blood of one or more healthy individuals, wherein a decrease in the magnitude of the level of TCP-1 gamma in the patients as compared to the levels of the healthy control individuals is diagnostic of ulcerative colitis. It is believed that measurement of the expression of the various subunits of TCP-1, such as TCP-1 gamma, will provide better sensitivity and specificity for diagnosing ulcerative colitis and distinguishing the disease from other colon diseases.

To detect an RNA transcript encoding TCP-1 gamma, nucleic acids can be isolated from a blood sample. The nucleic acid can be whole cell RNA or fractionated to Poly-A+. It may be desired to convert the RNA to a complementary DNA (cDNA). Normally, the nucleic acid is amplified. Methods for isolating, fractionating and amplifying nucleic acids from samples is well-known in the art. See, e.g., Sambrook and Russell (2001) supra and other standard laboratory molecular biology protocol manuals.

A variety of methods can be used to evaluate or quantitate the level of TCP-1 gamma RNA transcript present in the sample. For example, levels of TCP-1 gamma RNA transcript can be evaluated using well-known methods such as northern blot analysis (see, e.g., Sambrook and Russell (2001) supra); oligonucleotide or cDNA fragment hybridization wherein the oligonucleotide or cDNA is configured in an array on a chip or wafer; RNase protection analysis; or RT-PCR, as exemplified herein.

Suitable primers, probes, or oligonucleotides useful for such diagnostic methods can be generated by the skilled artisan from the sequence provided as GENBANK accession number NM_{—}005998. The term primer, as defined herein, is meant to encompass any nucleic acid that is capable of priming the synthesis of a nascent nucleic acid in a template-dependent process. Typically, primers are oligonucleotides from ten to twenty-five base pairs in length, but longer sequences can be employed. Primers can be provided in double-stranded or single-stranded form. Probes are defined differently, although they can act as primers. Probes, while perhaps capable of priming, are designed for hybridizing to the target DNA or RNA and need not be used in an amplification process. In particular embodiments, the probes or primers are labeled with, for example, radioactive species (³²P, ¹⁴C, ³⁵S, ³H, or other label) or a fluorophore (rhodamine, fluorescein). Depending on the application, the probes or primers can be used cold, i.e., unlabeled, and the RNA or cDNA molecules are labeled.

Various RT-PCR methodologies can be employed to evaluate the level of TCP-1 gamma RNA transcript present in a sample. As clinical samples are of variable quantity and quality a relative quantitative RT-PCR reaction can be performed with an internal standard. The internal standard can be an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

Other assays can be performed using a more conventional relative quantitative RT-PCR assay with an external standard protocol. These assays sample the PCR products in the linear portion of their amplification curves. The number of PCR cycles that are optimal for sampling must be empirically determined for each target cDNA fragment. In addition, the reverse transcriptase products of each RNA population isolated from the various samples must be carefully normalized for equal concentrations of amplifiable cDNAs. This consideration is very important since the assay measures absolute mRNA abundance. Absolute mRNA abundance can be used as a measure of differential gene expression only in normalized samples. While empirical determination of the linear range of the amplification curve and normalization of cDNA preparations are tedious and time consuming processes, the resulting RT-PCR assays can be superior to those derived from the relative quantitative RT-PCR assay with an internal standard.

Alternatively chip-based DNA technologies can be employed. Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see, e.g., Pease, et al. (1994) Proc. Natl. Acad. Sci. USA 91(11):5022-6; Fodor, et al. (1991) Science 251(4995):767-73).

Depending on the format, detection can be performed by visual means (e.g., ethidium bromide staining of a gel). Alternatively, detection can involve indirect identification of the product via chemiluminescence, radiolabel or fluorescent label or even via a system using electrical or thermal impulse signals (Bellus (1994) J. Macromol. Sci. Pure Appl. Chem. A311:1355-1376).

It is contemplated that the TCP-1 gamma protein levels are also elevated in patients with ulcerative colitis. Accordingly, in an alternative embodiment, TCP-1 gamma protein is detected in a sample. In general, the detection of TCP-1 gamma protein is carried out by immunoassays using antibodies which specifically bind to TCP-1 gamma. Antibodies which specifically bind TCP-1 gamma can be either polyclonal or monoclonal. Moreover, such antibodies can be natural or partially or wholly synthetically produced. All fragments or derivatives thereof which maintain the ability to specifically bind to TCP-1 gamma protein are also included. The antibodies can be a member of any immunoglobulin class, including any of the human classes: IgG, IgM, IgA, IgD, and IgE.

Antibody fragments can be any derivative of an antibody which is less than full-length. In general, an antibody fragment retains at least a significant portion of the full-length antibody's specific binding ability. Examples of antibody fragments include, but are not limited to, Fab, Fab′, F(ab′)₂, scFv, Fv, diabody, or Fd fragments. The antibody fragment can be produced by any means. For instance, the antibody fragment can be enzymatically or chemically produced by fragmentation of an intact antibody or it can be recombinantly produced from a gene encoding the partial antibody sequence. The antibody fragment can optionally be a single-chain antibody fragment. Alternatively, the fragment can be multiple chains which are linked together, for instance, by disulfide linkages. The fragment can also optionally be a multi-molecular complex. A functional antibody fragment typically contains at least about 50 amino acids and more typically contains at least about 200 amino acids.

An antibody for use in the methods of the present invention can be generated using classical cloning and cell fusion techniques. For example, the antigen of interest is typically administered (e.g., intraperitoneal injection) to wild-type or inbred mice (e.g., BALB/c) or transgenic mice which produce desired antibodies, or rats, rabbits or other animal species which can produce native or human antibodies. The antigen can be administered alone, or mixed with adjuvant, or expressed from a vector (VEE replicon vector), or as DNA, or as a fusion protein to induce an immune response. Fusion proteins contain the peptide against which an immune response is desired coupled to carrier proteins, such as histidine tag (his), mouse IgG2a Fc domain, β-galactosidase, glutathione S-transferase, keyhole limpet hemocyanin (KLH), or bovine serum albumin, to name a few. In these cases, the peptides serve as haptens with the carrier proteins. After the animal is boosted, for example, two or more times, the spleen is removed and splenocytes are extracted and fused with myeloma cells using the well-known processes (Kohler and Milstein (1975) Nature 256:495-497; Harlow and Lane (1988) Antibodies: A Laboratory Manual, Cold Spring Harbor Laboratory, New York). The resulting hybrid cells are then cloned in the conventional manner, e.g., using limiting dilution, and the resulting clones, which produce the desired monoclonal antibodies, are cultured.

Alternatively, antibodies which specifically bind TCP-1 gamma are produced by a phage display method. Methods of producing phage display antibodies are well-known in the art (e.g., Huse, et al. (1989) Science 246(4935):1275-81).

Selection of TCP-1 gamma-specific antibodies is based on binding affinity and can be determined by various well-known immunoassays including, enzyme-linked immunosorbent, immunodiffusion chemiluminescent, immunofluorescent, immunohistochemical, radioimmunoassay, agglutination, complement fixation, immunoelectrophoresis, and immunoprecipitation assays and the like which can be performed in vitro, in vivo or in situ. Such standard techniques are well-known to those of skill in the art (see, e.g., “Methods in Immunodiagnosis”, 2nd Edition, Rose and Bigazzi, eds. John Wiley & Sons, 1980; Campbell et al., “Methods and Immunology”, W. A. Benjamin, Inc., 1964; and Oellerich, M. (1984) J. Clin. Chem. Clin. Biochem. 22:895-904).

Anti-TCP-1 gamma antibodies can be used in diagnostic, prognostic, or predictive methods to evaluate the level of TCP-1 gamma in healthy and diseased samples via techniques such as ELISA, western blotting, or immunohistochemistry. The general method for detecting TCP-1 gamma provides contacting a sample with an antibody which specifically binds TCP-1 gamma. The antibody is allowed to bind to TCP-1 gamma to form an antibody-antigen complex. The conditions and time required to form the antibody-antigen complex may vary and are dependent on the sample being tested and the method of detection being used. Once non-specific interactions are removed by, for example, washing the sample, the antibody-antigen complex is detected using any one of the immunoassays described above as well a number of well-known immunoassays used to detect and/or quantitate antigens (see, for example, Harlow and Lane (1988) supra). Such well-known immunoassays include antibody capture assays, antigen capture assays, and two-antibody sandwich assays.

It is contemplated that the level of TCP-1 gamma expression may also correlate with the extent of disease and can be used in the prognosis of ulcerative colitis. It is further contemplated that one or more of the other subunits of TCP-1 may be expressed differentially in lymphocytes of inflammatory bowel disease patients and may differ between ulcerative colitis and Crohn's disease, thereby being useful as biomarkers for distinguishing ulcerative colitis from Crohn's disease.

The present invention is a method for identifying compounds useful for treating ulcerative colitis wherein the compounds identified are capable of decreasing the activity of TCP-1 gamma in cells or tissues. The method would comprise harvesting lymphocytes from the blood of patients with ulcerative colitis; determining a first level of TCP-1 gamma in the lymphocytes; contacting the lymphocytes with a compound; and determining a second level of TCP-1 gamma activity in the lymphocytes, wherein the compound is identified as being useful of treating ulcerative colitis if the magnitude of the second level of TCP-1 gamma is greater than the magnitude of the first level of TCP-1 gamma.

The following non-limiting examples are provided to further illustrate the present invention.

### EXAMPLES

### Example 1

### Real Time PCR and TCP-1 Expression

Amplification of cDNA by PCR involved making more copies of initial input cDNA using DNA polymerase and detecting the amount of resulting DNA by Sybergren dye at each cycle for 40 or 45 cycles. The number of the cycle at which there is a measurable DNA is called transition point (Cp) and is used to compare the relative levels of expression of the gene under consideration. More abundant genes will have low Cp whereas less abundant genes will have high Cp, when the same amount of initial cDNA is used in the PCR reaction. Normally, two reactions were done for each sample, with a 10-fold difference in the initial concentration of cDNA. Ideally, a difference in Cp for the same sample should be 3.3 when 10-fold dilutions are used. The amplification curve is obtained for multiple samples (e.g., healthy subjects and ulcerative patients are compared).

1. A composition for treating ulcerative colitis comprising interferon gamma, TCP-1 gamma and a pharmaceutically acceptable vehicle.

2. A method for decreasing the symptoms of ulcerative colitis in a patient comprising contacting cells or tissues of a patient with the composition of claim 1, and monitoring the level of TCP-1 gamma gene expression in cells or tissues, wherein an increase in the expression of TCP-1 gamma in said cells or tissues will result in a decrease in the symptoms of ulcerative colitis in said patient.

3. A method for diagnosing ulcerative colitis in a patient comprising:
a) determining a level of TCP-1 gamma in lymphocytes from blood of a patient suspected of having ulcerative colitis;
b) determining a level of TCP-1 gamma in lymphocytes from the blood of several healthy individuals; and
c) comparing the magnitude of the levels of TCP-1 gamma in said patient and said healthy control individuals, wherein a decrease in the magnitude of the level of TCP-1 gamma in the patient as compared to the levels of the healthy control individuals is diagnostic of ulcerative colitis.

4. A method for identifying compounds useful for treating ulcerative colitis comprising:
a) determining a first level of TCP-1 gamma in lymphocytes harvested from blood of patients with ulcerative colitis;
b) contacting the lymphocytes with a compound; and
c) determining a second level of TCP-1 gamma activity in the lymphocytes, wherein the compound is identified as being useful of treating ulcerative colitis if the magnitude of the second level of TCP-1 gamma is greater than the magnitude of the first level of TCP-1 gamma.

5. A method for evaluating efficacy of treatment of ulcerative colitis comprising:
a) determining a level of TCP-1 gamma in lymphocytes from blood of a patient before administration of an agent to treat ulcerative colitis;
b) administering to the patient an agent capable of treating ulcerative colitis;
c) determining a level of TCP-1 gamma in lymphocytes from the blood of the patient after administration of the agent; and
d) comparing the magnitude of the levels of TCP-1 gamma in said patient before and after administration of the agent, wherein an increase in the magnitude of the level of TCP-1 gamma in the patient after treatment with the agent as compared to the levels of TCP-1 gamma in lymphocytes before treatment with the agent is indicative of an effective treatment for ulcerative colitis.
